SEQUENCE LISTING (22 sequences)

SEQ ID NO: 1 - protein, 4544 aa, Homo sapiens
MLTPPLLLLLPLLSALVAAAIDAPKTCSPKQFACRDQITCISKGWRCDGERDCPDGSDEAPEICPQSKAQRCQPNEHNCLGTELCVPMSRLCNGVQDCMDGSDEGPHCRELQGNCSRLGCQHHCVPTLDGPTCYCNSSFQLQADGKTCKDFDECSVYGTCSQLCTNTDGSFICGCVEGYLLQPDNRSCKAKNEPVDRPPVLLIANSQNILATYLSGAQVSTITPTSTRQTTAMDFSYANETVCWVHVGDSAAQTQLKCARMPGLKGFVDEHTINISLSLHHVEQMAIDWLTGNFYFVDDIDDRIFVCNRNGDTCVTLLDLELYNPKGIALDPAMGKVFFTDYGQIPKVERCDMDGQNRTKLVDSKIVFPHGITLDLVSRLVYWADAYLDYIEVVDYEGKGRQTIIQGILIEHLYGLTVFENYLYATNSDNANAQQKTSVIRVNRFNSTEYQVVTRVDKGGALHIYHQRRQPRVRSHACENDQYGKPGGCSDICLLANSHKARTCRCRSGFSLGSDGKSCKKPEHELFLVYGKGRPGIIRGMDMGAKVPDEHMIPIENLMNPRALDFHAETGFIYFADTTSYLIGRQKIDGTERETILKDGIHNVEGVAVDWMGDNLYWTDDGPKKTISVARLEKAAQTRKTLIEGKMTHPRAIVVDPLNGWMYWTDWEEDPKDSRRGRLERAWMDGSHRDIFVTSKTVLWPNGLSLDIPAGRLYWVDAFYDRIETILLNGTDRKIVYEGPELNHAFGLCHHGNYLFWTEYRSGSVYRLERGVGGAPPTVTLLRSERPPIFEIRMYDAQQQQVGTNKCRVNNGGCSSLCLATPGSRQCACAEDQVLDADGVTCLANPSYVPPPQCQPGEFACANSRCIQERWKCDGDNDCLDNSDEAPALCHQHTCPSDRFKCENNRCIPNRWLCDGDNDCGNSEDESNATCSARTCPPNQFSCASGRCIPISWTCDLDDDCGDRSDESASCAYPTCFPLTQFTCNNGRCININWRCDNDNDCGDNSDEAGCSHSCSSTQFKCNSGRCIPEHWTCDGDNDCGDYSDETHANCTNQATRPPGGCHTDEFQCRLDGLCIPLRWRCDGDTDCMDSSDEKSCEGVTHVCDPSVKFGCKDSARCISKAWVCDGDNDCEDNSDEENCESLACRPPSHPCANNTSVCLPPDKLCDGNDDCGDGSDEGELCDQCSLNNGGCSHNCSVAPGEGIVCSCPLGMELGPDNHTCQIQSYCAKHLKCSQKCDQNKFSVKCSCYEGWVLEPDGESCRSLDPFKPFIIFSNRHEIRRIDLHKGDYSVLVPGLRNTIALDFHLSQSALYWTDVVEDKIYRGKLLDNGALTSFEVVIQYGLATPEGLAVDWIAGNIYWVESNLDQIEVAKLDGTLRTTLLAGDIEHPRAIALDPRDGILFWTDWDASLPRIEAASMSGAGRRTVHRETGSGGWPNGLTVDYLEKRILWIDARSDAIYSARYDGSGHMEVLRGHEFLSHPFAVTLYGGEVYWTDWRTNTLAKANKWTGHNVTVVQRTNTQPFDLQVYHPSRQPMAPNPCEANGGQGPCSHLCLINYNRTVSCACPHLMKLHKDNTTCYEFKKFLLYARQMEIRGVDLDAPYYNYIISFTVPDIDNVTVLDYDAREQRVYWSDVRTQAIKRAFINGTGVETVVSADLPNAHGLAVDWVSRNLFWTSYDTNKKQINVARLDGSFKNAVVQGLEQPHGLVVHPLRGKLYWTDGDNISMANMDGSNRTLLFSGQKGPVGLAIDFPESKLYWISSGNHTINRCNLDGSGLEVIDAMRSQLGKATALAIMGDKLWWADQVSEKMGTCSKADGSGSVVLRNSTTLVMHMKVYDESIQLDHKGTNPCSVNNGDCSQLCLPTSETTRSCMCTAGYSLRSGQQACEGVGSFLLYSVHEGIRGIPLDPNDKSDALVPVSGTSLAVGIDFHAENDTIYWVDMGLSTISRAKRDQTWREDVVTNGIGRVEGIAVDWIAGNIYWTDQGFDVIEVARLNGSFRYVVISQGLDKPRAITVHPEKGYLFWTEWGQYPRIERSRLDGTERVVLVNVSISWPNGISVDYQDGKLYWCDARTDKIERIDLETGENREVVLSSNNMDMFSVSVFEDFIYWSDRTHANGSIKRGSKDNATDSVPLRTGIGVQLKDIKVFNRDRQKGTNVCAVANGGCQQLCLYRGRGQRACACAHGMLAEDGASCREYAGYLLYSERTILKSIHLSDERNLNAPVQPFEDPEHMKNVIALAFDYRAGTSPGTPNRIFFSDIHFGNIQQINDDGSRRITIVENVGSVEGLAYHRGWDTLYWTSYTTSTITRHTVDQTRPGAFERETVITMSGDDHPRAFVLDECQNLMFWTNWNEQHPSIMRAALSGANVLTLIEKDIRTPNGLAIDHRAEKLYFSDATLDKIERCEYDGSHRYVILKSEPVHPFGLAVYGEHIFWTDWVRRAVQRANKHVGSNMKLLRVDIPQQPMGIIAVANDTNSCELSPCRINNGGCQDLCLLTHQGHVNCSCRGGRILQDDLTCRAVNSSCRAQDEFECANGECINFSLTCDGVPHCKDKSDEKPSYCNSRRCKKTFRQCSNGRCVSNMLWCNGADDCGDGSDEIPCNKTACGVGEFRCRDGTCIGNSSRCNQFVDCEDASDEMNCSATDCSSYFRLGVKGVLFQPCERTSLCYAPSWVCDGANDCGDYSDERDCPGVKRPRCPLNYFACPSGRCIPMSWTCDKEDDCEHGEDETHCNKFCSEAQFECQNHRCISKQWLCDGSDDCGDGSDEAAHCEGKTCGPSSFSCPGTHVCVPERWLCDGDKDCADGADESIAAGCLYNSTCDDREFMCQNRQCIPKHFVCDHDRDCADGSDESPECEYPTCGPSEFRCANGRCLSSRQWECDGENDCHDQSDEAPKNPHCTSPEHKCNASSQFLCSSGRCVAEALLCNGQDDCGDSSDERGCHINECLSRKLSGCSQDCEDLKIGFKCRCRPGFRLKDDGRTCADVDECSTTFPCSQRCINTHGSYKCLCVEGYAPRGGDPHSCKAVTDEEPFLIFANRYYLRKLNLDGSNYTLLKQGLNNAVALDFDYREQMIYWTDVTTQGSMIRRMHLNGSNVQVLHRTGLSNPDGLAVDWVGGNLYWCDKGRDTIEVSKLNGAYRTVLVSSGLREPRALVVDVQNGYLYWTDWGDHSLIGRIGMDGSSRSVIVDTKITWPNGLTLDYVTERIYWADAREDYIEFASLDGSNRHVVLSQDIPHIFALTLFEDYVYWTDWETKSINRAHKTTGTNKTLLISTLHRPMDLHVFHALRQPDVPNHPCKVNNGGCSNLCLLSPGGGHKCACPTNFYLGSDGRTCVSNCTASQFVCKNDKCIPFWWKCDTEDDCGDHSDEPPDCPEFKCRPGQFQCSTGICTNPAFICDGDNDCQDNSDEANCDIHVCLPSQFKCTNTNRCIPGIFRCNGQDNCGDGEDERDCPEVTCAPNQFQCSITKRCIPRVWVCDRDNDCVDGSDEPANCTQMTCGVDEFRCKDSGRCIPARWKCDGEDDCGDGSDEPKEECDERTCEPYQFRCKNNRCVPGRWQCDYDNDCGDNSDEESCTPRPCSESEFSCANGRCIAGRWKCDGDHDCADGSDEKDCTPRCDMDQFQCKSGHCIPLRWRCDADADCMDGSDEEACGTGVRTCPLDEFQCNNTLCKPLAWKCDGEDDCGDNSDENPEECARFVCPPNRPFRCKNDRVCLWIGRQCDGTDNCGDGTDEEDCEPPTAHTTHCKDKKEFLCRNQRCLSSSLRCNMFDDCGDGSDEEDCSIDPKLTSCATNASICGDEARCVRTEKAAYCACRSGFHTVPGQPGCQDINECLRFGTCSQLCNNTKGGHLCSCARNFMKTHNTCKAEGSEYQVLYIADDNEIRSLFPGHPHSAYEQAFQGDESVRIDAMDVHVKAGRVYWTNWHTGTISYRSLPPAAPPTTSNRHRRQIDRGVTHLNISGLKMPRGIAIDWVAGNVYWTDSGRDVIEVAQMKGENRKTLISGMIDEPHAIVVDPLRGTMYWSDWGNHPKIETAAMDGTLRETLVQDNIQWPTGLAVDYHNERLYWADAKLSVIGSIRLNGTDPIVAADSKRGLSHPFSIDVFEDYIYGVTYINNRVFKIHKFGHSPLVNLTGGLSHASDVVLYHQHKQPEVTNPCDRKKCEWLCLLSPSGPVCTCPNGKRLDNGTCVPVPSPTPPPDAPRPGTCNLQCFNGGSCFLNARRQPKCRCQPRYTGDKCELDQCWEHCRNGGTCAASPSGMPTCRCPTGFTGPKCTQQVCAGYCANNSTCTVNQGNQPQCRCLPGFLGDRCQYRQCSGYCENFGTCQMAADGSRQCRCTAYFEGSRCEVNKCSRCLEGACVVNKQSGDVTCNCTDGRVAPSCLTCVGHCSNGGSCTMNSKMMPECQCPPHMTGPRCEEHVFSQQQPGHIASILIPLLLLLLLVLVAGVVFWYKRRVQGAKGFQHQRMTNGAMNVEIGNPTYKMYEGGEPDDVGGLLDADFALDPDKPTNFTNPVYATLYMGGHGSRHSLASTDEKRELLGRGPEDEIGDPLA

SEQ ID NO: 2 - protein, 331 aa, Artificial Sequence
  synthetic cluster II (LRPII)
PQCQPGEFACANSRCIQERWKCDGDNDCLDNSDEAPALCHQHTCPSDRFKCENNRCIPNRWLCDGDNDCGNSEDESNATCSARTCPPNQFSCASGRCIPISWTCDLDDDCGDRSDESASCAYPTCFPLTQFTCNNGRCININWRCDNDNDCGDNSDEAGCSHSCSSTQFKCNSGRCIPEHWTCXGDNDCGDYSDETHANCTNQATRPPGGCHTDEFQCRLDGLCIPLRWRCDGDTDCMDSSDEKSCEGVTHVCDPSVKFGCKDSARCISKAWVCDGDNDCEDNSDEENCESLACRPPSHPCANNTSVCLPPDKLCDGNDDCGDGSDEGELC

SEQ ID NO: 3 - protein, 447 aa, Artificial Sequence
  synthetic cluster IV (LRPIV)
SNCTASQFVCKNDKCIPFWWKCDTEDDCGDHSDEPPDCPEFKCRPGQFQCSTGICTNPAFICDGDNDCQDNSDEANCDIHVCLPSQFKCTNTNRCIPGIFRCNGQDNCGDGEDERDCPEVTCAPNQFQCSITKRCIPRVWVCDRDNDCVDGSDEPANCTQMTCGVDEFRCKDSGRCIPARWKCDGEDDCGDGSDEPKEECDERTCEPYQFRCKNNRCVPGRWQCDYDNDCGDNSDEESCTPRPCSESEFSCANGRCIAGRWKCDGDHDCADGSDEKDCTPRCDMDQFQCKSGHCIPLRWRCDADADCMDGSDEEACGTGVRTCPLDEFQCNNTLCKPLAWKCXGEDDCGDNSDENPEECARFVCPPNRPFRCKNDRVCLWIGRQCDGTDNCGDGTDEEDCEPPTAHTTHCKDKKEFLCRNQRCLSSSLRCNMFDDCGDGSDEEDCSI

SEQ ID NO: 4 - protein, 41 aa, Artificial Sequence
  synthetic complement repeat motif (CR3)
PQCQPGEFACANSRCIQERWKCXGDNDCLDNSDEAPALCHQ

SEQ ID NO: 5 - protein, 41 aa, Artificial Sequence
  synthetic complement repeat motif (CR4)
HTCPSDRFKCENNRCIPNRWLCXGDNDCGNSEDESNATCSA

SEQ ID NO: 6 - protein, 40 aa, Artificial Sequence
  synthetic complement repeat motif (CR5)
RTCPPNQFSCASGRCIPISWTCDLDDDCGDRSDESASCAY

SEQ ID NO: 7 - protein, 40 aa, Artificial Sequence
  synthetic complement repeat motif (CR6)
PTCFPLTQFTCNNGRCININWRCDNDNDCGDNSDEAGCSH

SEQ ID NO: 8 - protein, 40 aa, Artificial Sequence
  synthetic complement repeat motif (CR7)
SCSSTQFKCNSGRCIPEHWTCXGDNDCGDYSDETHANCTN

SEQ ID NO: 9 - protein, 46 aa, Artificial Sequence
  synthetic complement repeat motif (CR8)
QATRPPGGCHTDEFQCRLDGLCIPLRWRCDGDTDCMDSSDEKSCEG

SEQ ID NO: 10 - protein, 43 aa, Artificial Sequence
  synthetic complement repeat motif (CR9)
VTHVCDPSVKFGCKDSARCISKAWVCDGDNDCEDNSDEENCES

SEQ ID NO: 11 - protein, 40 aa, Artificial Sequence
  synthetic complement repeat motif (CR10)
LACRPPSHPCANNTSVCLPPDKLCDGNDDCGDGSDEGELC

SEQ ID NO: 12 - protein, 38 aa, Artificial Sequence
  synthetic complement repeat motif (CR21)
SNCTASQFVCKNDKCIPFWWKCXTEDDCGDHSDEPPDC

SEQ ID NO: 13 - protein, 39 aa, Artificial Sequence
  synthetic complement repeat motif (CR22)
PEFKCRPGQFQCSTGICTNPAFICXGDNDCQDNSDEANC

SEQ ID NO: 14 - protein, 42 aa, Artificial Sequence
  synthetic complement repeat motif (CR23)
DIHVCLPSQFKCTNTNRCIPGIFRCNGQDNCGDGEDERDCPE

SEQ ID NO: 15 - protein, 41 aa, Artificial Sequence
  synthetic complement repeat motif (CR24)
VTCAPNQFQCSITKRCIPRVWVCXRDNDCVDGSDEPANCTQ

SEQ ID NO: 16 - protein, 40 aa, Artificial Sequence
  synthetic complement repeat motif (CR25)
MTCGVDEFRCKDSGRCIPARWKCXGEDDCGDGSDEPKEEC

SEQ ID NO: 17 - protein, 39 aa, Artificial Sequence
  synthetic complement repeat motif (CR26)
DERTCEPYQFRCKNNRCVPGRWQCXYDNDCGDNSDEESC

SEQ ID NO: 18 - protein, 39 aa, Artificial Sequence
  synthetic complement repeat motif (CR27)
TPRPCSESEFSCANGRCIAGRWKCXGDHDCADGSDEKDC

SEQ ID NO: 19 - protein, 38 aa, Artificial Sequence
  synthetic complement repeat motif (CR28)
TPRCDMDQFQCKSGHCIPLRWRCXADADCMDGSDEEAC

SEQ ID NO: 20 - protein, 43 aa, Artificial Sequence
  synthetic complement repeat motif (CR29)
GTGVRTCPLDEFQCNNTLCKPLAWKCXGEDDCGDNSDENPEEC

SEQ ID NO: 21 - protein, 41 aa, Artificial Sequence
  synthetic complement repeat motif (CR30)
ARFVCPPNRPFRCKNDRVCLWIGRQCXGTDNCGDGTDEEDC

SEQ ID NO: 22 - protein, 47 aa, Artificial Sequence
  synthetic complement repeat motif (CR31)
EPPTAHTTHCKDKKEFLCRNQRCLSSSLRCNMFDDCGDGSDEEDCSI

(19) United States
(12) Patent Application Publication
(10) Pub. No.: US 2012/0201757 A1
(43) Pub. Date: Aug. 9, 2012

(54) MUTANT LOW-DENSITY LIPOPROTEIN RECEPTOR RELATED PROTEIN WITH INCREASED BINDING TO ALZHEIMER AMYLOID-BETA PEPTIDE

(76) Inventors: Berislav V. Zlokovic, Rochester, NY (US); Alaka Srivastava, Solon, OH (US); Rashid Deane, Rochester, NY (US)

(21) Appl. No.: 13/501,877

(22) PCT Filed: Oct. 18, 2010
(86) PCT No.: PCT/US2010/002769

§ 371 Date: Apr. 13, 2012

Related U.S. Application Data
(60) Provisional application No. 61/252,670, filed on Oct. 17, 2009.

(51) Int. Cl. A61K 49/00; A61P 25/28; A61K 38/17; G01N 33/566; C07K 14/705; C07K 19/00

(52) U.S. Cl. 424/9.1; 530/350; 514/1.1; 436/501; 435/7.1; 530/402

(57) ABSTRACT

A mutant low-density lipoprotein receptor related protein-1 binds to Alzheimer amyloid-beta (Aβ) peptide with greater affinity compared to its wild-type homolog. This binding may be used to detect Aβ or to separate Aβ from the rest of a subject's body. In Alzheimer disease, it may be used to provide diagnostic results by detecting Aβ, treatment by removing Aβ, or both.

### FIELD OF THE INVENTION

The invention relates to mutation of the low-density lipoprotein receptor related protein-1 to improve its binding to Alzheimer amyloid-beta (Aβ) peptide. This specific binding may be used to detect Aβ or to separate Aβ from the rest of a subject's body. In Alzheimer disease, the invention may be used to provide diagnostic results by detecting Aβ, treatment by removing Aβ, or both.

### BACKGROUND OF THE INVENTION

Amyloid-beta (Aβ) peptide is known to be involved in the pathology of Alzheimer disease (AD). This peptide is the main constituent of amyloid in the brain parenchyma and vasculature. Aβ extracted from senile plaques is mainly peptides Aβ₁₋₄₀ (Aβ40) and Aβ₁₋₄₂ (Aβ42); vascular amyloid is mainly peptides Aβ₁₋₃₉ and Aβ40. The major soluble form of Aβ present in blood, cerebrospinal fluid (CSF), and brain is Aβ40. Soluble Aβ which is circulating in blood, CSF, and brain interstitial fluid (ISF) may exist as free peptide and/or associated with apolipoprotein E (apoE), apolipoprotein J (apoJ), other lipoproteins, albumin, α2-macroglobulin (α₂M), and transthyretin.

According to the amyloid hypothesis, accumulation of neurotoxic Aβ42 in the brain is a major event initiating Aβ pathogenesis (Hardy & Selkoe, 2002). Increased Aβ42 accumulation could be associated with increased production of Aβ as in familial forms of Aβ and/or impaired clearance of Aβ as in a late-onset AD (Selkoe, 2001; Zlokovic & Frangione, 2003). Increased levels of Aβ in the brain results in formation of neurotoxic Aβ oligomers and progressive synaptic, neuritic, and neuronal dysfunction (Walsh et al., 2002; Dahlgren et al., 2002; Kayed et al., 2003; Gong et al., 2003). Missense mutations within Aβ associate mainly with vascular deposits, as in patients with Dutch mutation (G to C at codon 693, Glu to Gln at position 22) and Iowa mutation (G to A at codon 694, Asp to Asn at position 23). Vasculotropic Dutch (E22Q) or Iowa (D23N) mutant Aβ exhibit enhanced fibrillogenesis and toxicity to cerebral vascular cells, while Dutch/Iowa double mutant Aβ (E22Q,D23N) has accelerated pathogenic properties compared to both Dutch and Iowa vasculotropic mutants (Van Nostrand et al., 2001).

Cell surface proteins such as the receptor for advanced glycation end products (RAGE), scavenger type A receptor (SR-A), native LRP-1, and LRP-2 bind Aβ at low nanomolar concentrations as free peptide (e.g., RAGE, SR-A), and/or in complex with apoE, apoJ, or α₂M (e.g., native LRP-1, LRP-2). But mutant LRP-1 that directly binds to Aβ with greater affinity than its wild-type homolog was not disclosed.

WO 01/90758 and US 2004/0259159 describe LRP-1's role in mediating vascular clearance of Aβ from the brain. It was taught that increasing LRP-1 expression or activity can be used to remove Aβ, and thereby treat a subject with Alzheimer disease or at risk for developing the disease.

WO 2005/122712 and US 2007/0054318 describe the use of a soluble LRP-1 to bind Aβ and remove it from the brain. Soluble cluster II or IV of LRP-1 (LRPII or LRPIV, respectively) was shown to bind Aβ in vitro and in vivo with one- to two-orders of magnitude greater affinity than other known ligands (e.g., tPA, apoE2, apoE3, apoE4, MMP9). In vivo, wild-type (wt) cluster IV of LRP-1 (wt-LRPIV) exerts a strong Aβ peripheral sink activity, which results in Aβ clearance from brain that significantly reduces amyloid-related pathology and improves functional outcome in transgenic mice. Aβ-precursor protein (APP), an APP770 isoform with a Kunitz-type protease inhibitor (KPI) domain, but not a shorter APP695 (the most common APP isoform in brain which lacks the KPI domain), was shown to bind to LRP-1 in vitro resulting in APP degradation in cultured fibroblasts. See Deane et al. (2004) and Sagare et al. (2007).

Here, we show that APP695 does not bind to wt-LRPIV. Moreover, APP isoforms containing a KPI domain (e.g., APP770, APP751, and sAPPβ) do not detectably bind wt-LRPIV at an Aβ binding site. KPI-containing APP isoforms did exhibit very weak binding for wt-LRPIV that was two orders of magnitude lower than for Aβ. This weak binding of KPI-containing APP isoforms to wt-LRPIV was abolished with a KPI-specific anti-body or a recombinant KPI peptide, which did not affect Aβ binding (i.e., the binding was not specific for the Aβ binding pocket of LRP-1). We found that mutant LRP-1, which contains a single mutation at residue 343 of aspartic acid to glycine (D343G), bound Aβ42 with a three-fold greater affinity than wt-LRPIV (Kd˜1.5 nM) and exerted a significantly greater by 30-50% Aβ peripheral sink action in control mice for Aβ40 and Aβ42 than wt-LRPIV. Further, mutant LRPIV did not detectably bind KPI-containing APP isoforms (i.e., APP770, APP751, and sAPPβ) and did not cross the blood-brain barrier. Both mutant LRPIV and wt-LRPIV failed to alter APP levels and/or metabolism in brain. Thus, mutant LRP-1 with greater binding affinity for Aβ than wt-LRPIV can be used as a specific Aβ sink agent without any significant affect on APP metabolism in brain or periphery.

Mutant LRP-1 proteins and nucleic acids encoding them, medicaments and compositions, and their use in methods of treatment and diagnosis are taught herein to be applicable to formation of amyloid and its role in disease. Importantly, mutant LRP-1 acts as a sink in the periphery for depletion of Aβ from the central nervous system across the blood-brain barrier. Other advantages of the invention are discussed below or would be apparent to a person skilled in the art from that discussion.

### SUMMARY OF THE INVENTION

An objective is to improve binding affinity to an amyloid-beta (Aβ) peptide by mutation of low-density lipoprotein receptor related protein-1 (LRP-1). As compared to binding by native LRP-1, it is preferred that the mutant LRP-1 has greater affinity for specifically binding Aβ. Further, it is preferred that a derivative of LRP-1, which comprises at least a mutation of aspartic acid in one or more calcium-binding fragments, binds Aβ with at least two-fold greater affinity than a derivative of LRP-1 that comprises all mutations except for not substituting wild-type aspartic acid in the one or more calcium-binding fragments. More preferably, the substitution of aspartic acid results in at least three-fold, at least four-fold, at least five-fold, at least six-fold, at least seven-fold, at least eight-fold, at least nine-fold, or at least ten-fold greater affinity for binding Aβ.

In one embodiment, a mutant LRP-1 is provided. The mutant LRP-1 may be comprised of one or more domains derived from LRP-1 and, optionally, one or more domains not derived from LRP-1 (i.e., heterologous domains which do not exist in the native protein). It is preferred that at least cluster II and/or cluster IV is contained therein; it may consist essentially of only cluster II and/or cluster IV. More preferably, it contains at least cluster IV or consists essentially of only cluster IV; it may not contain cluster II or cluster IV when the other is present. The mutant LRP-1 may or may not contain other optional domains: a signal peptide that directs secretion out of the cell (e.g., a hydrophobic amino acid sequence targeting nascent polypeptide to endoplasmic reticulum, trans-locates polypeptide across the membrane, and transports polypeptide with any modifications through the secretory pathway) and a domain which attaches a polypeptide to a lipid bilayer (e.g., a transmembrane domain for docking across or a lipid domain for insertion into the membrane). A soluble LRP-1 mutant is preferred for binding Aβ in solution, probably by removing at least the trans-membrane domain of the native protein. The mutant LRP-1 may be reversibly or irreversibly attached to a solid substrate (e.g., using a covalent bond which is chemically labile or stable, respectively). It is not identical to native LRP-1 so one or more domains of the native amino acid sequence must be mutated (e.g., substitution, addition, deletion) while improving its ability to bind Aβ (e.g., preferably at least two-fold better binding compared to an equivalent protein not having the mutation). It is also preferred that human or another mammal be used as the source, and an undetectable immune response be elicited in the subject in whom the mutant LRP-1 is administered (e.g., derived from human or a humanized mammalian LRP-1 mutant infused into a human patient).

Mutant LRP-1 may be used in treatment as a medicament (e.g., therapy in a subject having the disease or prophylaxis in a subject at risk for developing the disease) or diagnosis as a direct binding agent for detection of Aβ. A therapeutic or prophylactic composition is comprised of mutant LRP-1 and at least one pharmaceutically-acceptable carrier (e.g., a solution of physiological salt and buffer). It may inactivate Aβ by removing Aβ from the subject through the body's circulatory systems or by machine (e.g., apheresis or other extracorporeal technology to form a mutant LRP-1/Aβ complex and remove the complex from the body), or by reducing deposition of amyloid. A diagnostic composition is comprised of mutant LRP-1 and at least one detectable label (e.g., a moiety for chromatic, enzymatic, fluorescent, luminescent, magnetic or paramagnetic, or radioactive detection). The mutant LRP-1 and the detectable label may or may not be covalently attached. Alternatively, they may be attached though one or more specific binding pairs. Binding may occur inside or outside the subject's body, in solution or with one of them immobilized on a substrate. Mutant LRP-1 directly bound to Aβ may be detected in a specimen prepared from a body fluid or tissue using a laboratory assay (i.e., in vitro diagnostics) or in the subject's body by fluoroscopic, magnetic resonance, or radiographic imaging (i.e., in vivo diagnostics). The subject may be a mammal, preferably a human.

Further aspects of the invention will be apparent to a person skilled in the art from the following detailed description and claims, and generalizations thereto.

### BRIEF DESCRIPTION OF THE DRAWINGS

FIG. 1 shows the amino acid sequence of LRP-1 (SEQ ID NO:1). At least a D184G mutation in a calcium-binding fragment (i.e., complement repeat motif CR7) of an LRPII minireceptor consists essentially of cluster II (SEQ ID NO:2) derived from LRP-1. At least a D343G mutation in a calcium-binding fragment (i.e., complement repeat motif CR29) of an LRPIV minireceptor consists essentially of cluster IV (SEQ ID NO:3) derived from LRP-1. Only some of the complement repeat motifs (CR3-CR10 of LRPII and CR21-CR31 of LRPIV, SEQ ID NOS:4-22) are calcium-binding fragments, in which mutation of aspartic acid would affect binding of a minireceptor to Aβ peptide. Aspartic acid follows cysteine in the calcium-binding fragments that would be engineered to affect binding of Aβ peptide: i.e., CR3 (SEQ ID NO:4), CR4 (SEQ ID NO:5), CR7 (SEQ ID NO:8), CR21 (SEQ ID NO:12), CR22 (SEQ ID NO:13), CR24 (SEQ ID NO:15), CR25 (SEQ ID NO:16), CR26 (SEQ ID NO:17), CR27 (SEQ ID NO:18), CR28 (SEQ ID NO:19), CR29 (SEQ ID NO:20), and CR30 (SEQ ID NO:21). Substitution of aspartic acid (D) with glycine (G) is preferred. Other possible substitutions that can be made at those positions are alanine (A), serine (S), and threonine (T).

FIG. 2 shows that LRPIV fragments bind with high affinity to Aβ. Graphs are binding curves for LRPIV fragments at different levels of human Aβ40 (FIG. 2A) and Aβ42 (FIG. 2B). Binding constants (Kd) are shown for the fragments binding to Aβ40 (FIG. 2C) and Aβ42 (FIG. 2D). Values are mean±s.e.m., n=3 assays per group.

FIG. 3 shows that mutant LRPIV binds to Aβ with higher affinity than other ligands of LRP-1. Graphs are binding curves for human apoE2 (E2), apoE3 (E3), apoE4 (E4), tPA, MMP9, and factor IXa to immobilized MT007-LRPIV (FIG. 3A) and GAR-LRPIV (FIG. 3B). Kd's are shown for the ligands binding to MT007-LRPIV (FIG. 3C) and GAR-LRPIV (FIG. 3D). Values are mean±s.e.m., n=3 assays per group.

FIG. 4 shows that mutant LRPIV binds to APP with lower affinity compared to wild-type LRPIV. Graphs are binding curves for APP695 to immobilized GAR-LRPIV (FIG. 4A), APP770 (FIG. 4B) and APP751 (FIG. 4C) to immobilized GAR-LRPIV in the absence and presence of soluble KPI (Kunitz protease inhibitor) domain and anti-KPI antibody (mAb 4.1), and Aβ40 (FIG. 4D) and Aβ42 (FIG. 4E) to immobilized GAR-LRPIV in the absence and presence of soluble KPI domain and mAb 4.1. Kd's for Aβ40, Aβ42, APP770, and APP751 binding to GAR-LRPIV are shown in FIG. 4F. Kd's for APP770 binding to immobilized GAR-LRPIV and MT007-LRPIV are compared in FIG. 4G. Values are mean±s.e.m., n=3 assays per group.

FIG. 5 shows that mutant LRPIV has greater potential for lowering brain Aβ than wild-type LRPIV. The levels of plasma Aβ40 (FIG. 5A), plasma Aβ42 (FIG. 5B), brain Aβ40 (FIG. 5C), and brain Aβ42 (FIG. 5D) are shown after treatment. Control (i.e., wild-type) mice were treated with vehicle and GAR-LRPIV or MT007-LRPIV (intravenously, 20 μg/day) for five days. At the end of the treatment period, plasma and brain samples of the mice were collected. Aβ levels were determined by ELISA. Values are mean±s.e.m., n=3 mice per group.

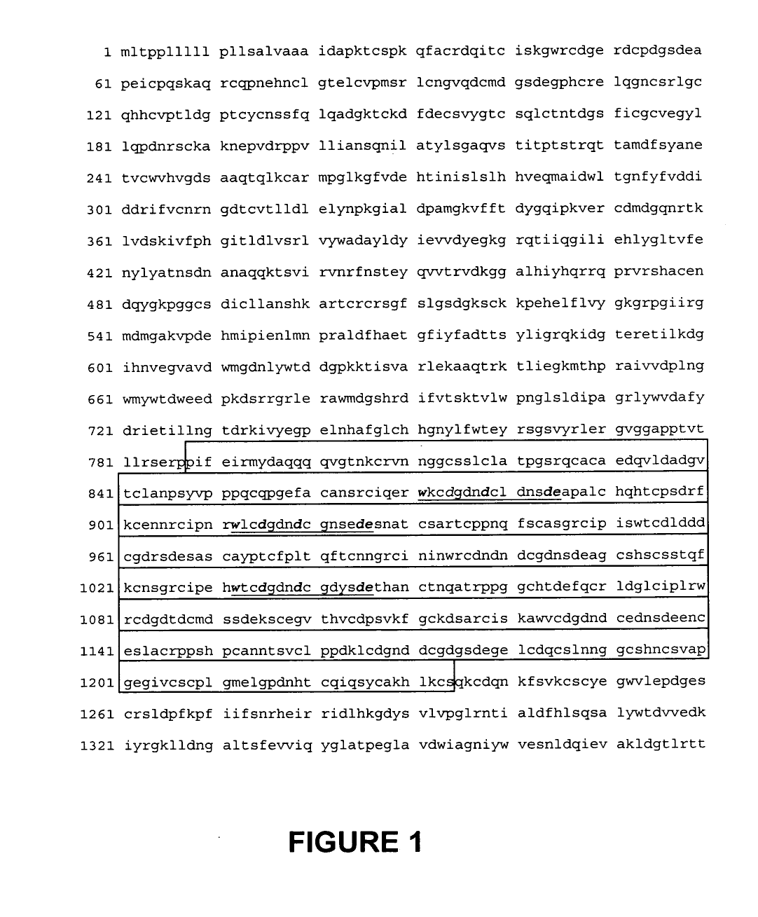

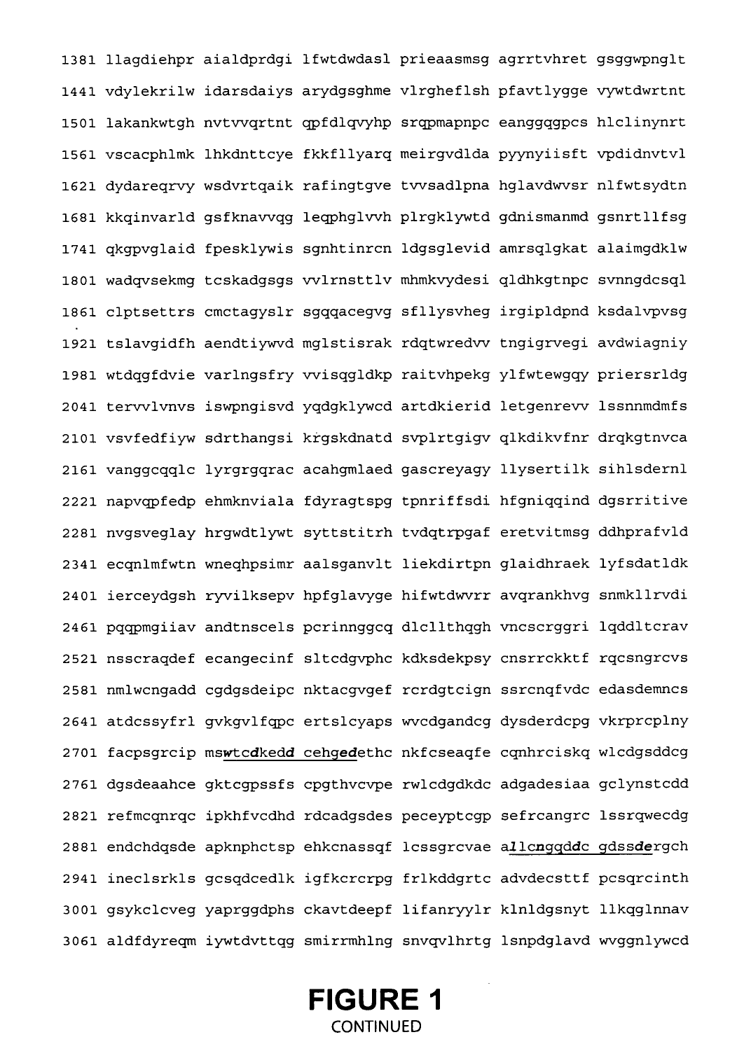

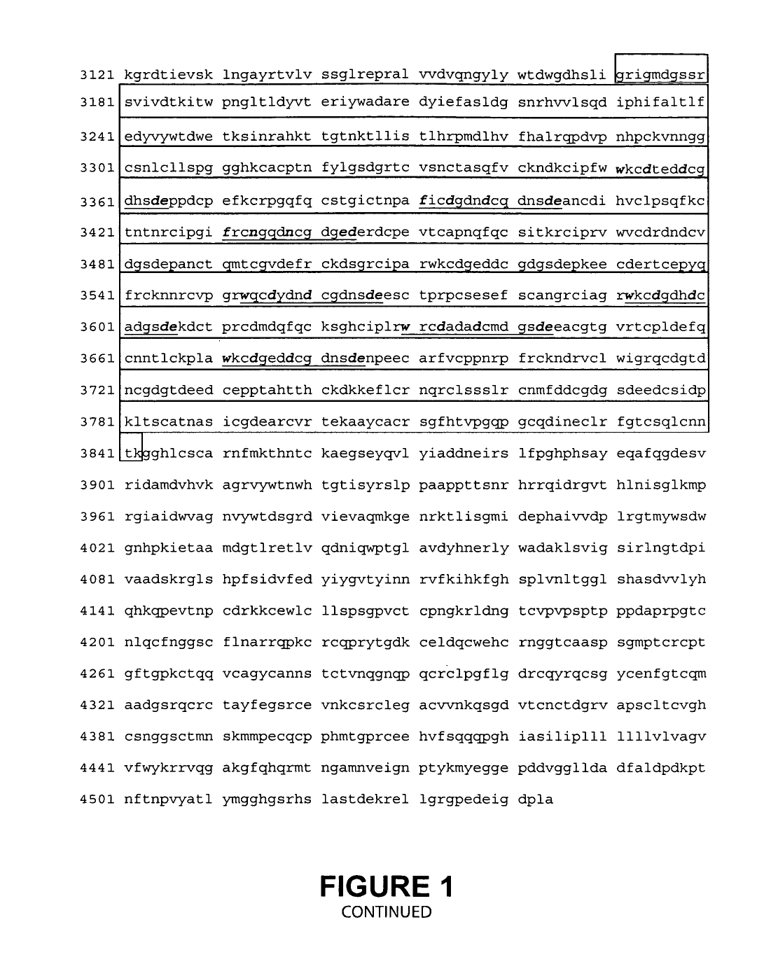

### DESCRIPTION OF SPECIFIC EMBODIMENTS OF THE INVENTION

Mature low-density lipoprotein receptor related protein-1 (LRP-1) is comprised of at least five different types of domains: (i) ligand-binding cysteine-rich repeats, (ii) epidermal growth factor (EGF) receptor-like cysteine-rich repeats, (iii) YWTD repeats, (iv) a transmembrane domain, and (v) a cytoplasmic domain. The signal peptide is cleaved after translocation into the secretory pathway. Ligand-binding-type domains in LRP-1 occur in four clusters (clusters I to IV) containing between two and eleven fragments. Most of the ligands for LRP-1 that have had their binding sites mapped interact with these ligand-binding-type domains (clusters II and IV, individually or together, contribute to the binding of Aβ peptide). They are followed by EGF precursor homology domains, which are comprised of two EGF repeats, six YWTD repeats arranged in a propeller-like structure, and another EGF repeat. Six EGF repeats precede the transmembrane domain. The cytoplasmic domain is comprised of two NPxY repeats that serve as docking sites for the endocytosis machinery and for cytoplasmic adaptor and scaffolding proteins which are involved in cell signaling. The heavy chain of LRP-1 (515 kDa) contains the four ligand-binding domains and the light chain of LRP-1 (85 kDa) contains the transmembrane and cytoplasmic domains. A mutant LRP-1 may be comprised of only the heavy chain or a fragment thereof. For a soluble LRP-1, the protein may lack the transmembrane domain and, preferably, the cytoplasmic domain as well.

LRP-1 recognizes at least 30 different ligands which represent several families of proteins, which include lipoproteins, proteinases, proteinase-inhibitor complexes, extracellular matrix (ECM) proteins, bacterial toxins, viruses, and various other intracellular proteins. The largest group of ligands recognized by LRP-1 are proteinases or molecules associated with regulating proteolytic activity. Certain serine proteinases and metalloproteinases bind directly to LRP-1, while a number of other proteinases only bind once complexed with their specific inhibitors. These inhibitors are only recognized by LRP-1 following a conformation change that occurs in them after proteolytic cleavage or reaction with small amines. In contrast, LRP-1 recognizes both the native and complexed forms of tissue factor pathway inhibitor (TFPI). LRP-1 also binds to the multimeric matrix proteins thrombospondin-1 and thrombospondin-2 and delivers Pseudomonas exotoxin A and minor-group human rhinovirus into cells. In addition, LRP-1 recognizes a number of intracellular proteins, including HSP96, HIV-1 Tat protein, and RAP, an endoplasmic reticulum resident protein that functions as a molecular chaperone for LRP-1 and other LDL receptor family members.

How does LRP-1 specifically recognize this variety of ligands? Crystallography and nuclear magnetic resonance of individual ligand-binding domains have revealed that amino acid sequence variability in short loops of each ligand-binding domain results in a unique contour surface and charge density for the repeats. LRP-1 “mini-receptors” have been made by fusing different ligand-binding domains to the LRP-1 light chain and measuring the ability to mediate the endocytosis of individual ligands following expression in cells. Preferably, soluble LRP-1 fragments may be made by recombinant technology and the different ligand-binding domains are screened for their ability to bind different ligands in vitro. Here, we demonstrate the role of calcium-binding fragments within the ligand-binding domain (see cluster IV) in specific binding of Aβ. They might act cooperatively to coordinate binding of calcium and Aβ peptide. Thus, Aβ binding may be grafted onto a heterologous polypeptide (cf humanization of rodent antibodies to reduce their immunogenicity) to make a mutant LRP-1.

A “fragment” is a particular mutation of LRP-1 with a molecular weight less than the molecular weight of full-length LRP-1. The molecular weight of mutant LRP-1's amino acid sequence is may be between the molecular weight of a single ligand-binding domain and the heavy chain of LRP-1 (515 kDa). For example, mutant LRP-1 may be from about 30 kDa to about 55 kDa, but both smaller and larger fragment are possible. In particular, cluster II (SEQ ID NO:2) and/or cluster IV (SEQ ID NO:3) of soluble LRP-1, or one or more calcium-binding fragments thereof are preferred. Thus, mutant LRP-1 having a relative molecular weight of less than about 65 kDa (primary amino acid sequence plus glycosylation) is possible. By contrast, exclusion of either cluster II or cluster IV is preferred from the mutant LRP-1 (i.e., comprising only cluster IV or cluster II, respectively) when minimizing a mutant's molecular weight is desirable. Wild-type LRP-1 protein and nucleic acid encoding the protein, its amino acid and nucleotide sequences, or its mature form may be derived from human (e.g., accession CAA32112, NP_{—}002323, Q07954, or S02392), other mammals (e.g., cow, guinea pig, mouse, rat), or polymorphisms and variants thereof. Although native LRP-1 protein might be chemically manipulated (e.g., hydrolytic cleavage or enzymatic proteolysis) to make polypeptide fragments, genetic manipulation of polynucleotides to make those fragments by recombinant technology in a bacterium, mold or yeast, insect, or mammalian cell or organism is preferred. A genetic chimera may be used to fuse a mutant LRP-1 to one or more heterologous domains. Nucleic acid encoding mutant LRP-1 may be introduced into cultured cells or organisms (e.g., nuclear transfer, transfection, transgenesis, especially into stem cells within or implanted into the body) where the polypeptide is translated and processed. For example, mutant LRP-1 protein may be produced from an expression construct introduced into cells by viral infection or transfection. Expression constructs preferably are transcribed from a regulatory region (e.g., promoter, enhancer) which is vascular cell-specific or derived from a virus, or a combination thereof. They may be associated with proteins and other nucleic acids in a carrier (e.g., packaged in a viral particle derived from an adenovirus, adeno-associated virus, cytomegalovirus, herpes simplex virus, or retrovirus, encapsulated in a liposome, or complexed with polymers). In vivo treatment includes instillation of a pharmaceutical composition (e.g., virus- or nucleic acid-containing solution) directly into vasculature of a subject. For ex vivo treatment, cells from a subject or donor (e.g., vascular cells or progenitors thereof) may be virally infected or transfected in vitro and then transplanted into vasculature of the subject. Cells may be vascular cells (e.g., smooth muscle cells), especially of brain, artery, or an organ of the reticuloendothelial system, and more especially of the cerebral artery at the blood-brain barrier, liver, or stem cells.

A preferred method of making a soluble LRP-1 involves mutating the wild-type transmembrane domain (e.g., a missense or deletion mutation). For example, a stop codon may be introduced at a site before the transmembrane domain or the portion encoding the transmembrane and cytoplasmic domains may be deleted. A mini-receptor comprising cluster IV or several calcium-binding fragments thereof may also be produced (e.g., by gene splicing or amplifying with adapter primers) and used for Aβ binding. Mutant LRP-1 may be attached to the lipid bilayer of a cellular membrane or another substrate, and then detached/hydrolyzed to make the mutant LRP-1. For example, a proteolytic enzyme may hydrolyze a peptide bond on the outside of a cell or a lipase may hydrolyze a glycosphingolipid anchor inserted in the lipid bilayer. Alternatively, mutant LRP-1 may or may not be immobilized on a substrate before, during, or after binding to Aβ.

Protein fusions may also be made and used. The native LRP-1 signal peptide or a heterologous signal peptide may be used to translocate the protein across the ER membrane and to transport it through the secretory pathway. Mutant LRP-1 may be glycosylated or otherwise post-translationally modified. A localization domain (e.g., antibody or another member of a binding pair) may be used to increase the local concentration of a soluble LRP-1 mutant in a tissue, organ, or other portion of a subject's body. For example, biotinylation or a fusion with streptavidin may localize the soluble LRP-1 mutant to a body part in/or which the cognate binding member (avidin or biotin, respectively) is attached.

For example, at least a mutation in the calcium-binding fragment may be made in any member of the LRP superfamily from human or other mammals (e.g., cow, guinea pig, mouse, or rat), especially LRP-1 homologs. The amino acid or nucleotide sequence of the mutant LRP-1 homolog may also include other known substitutions, deletions, insertions, fusions of heterologous domains, variants, or polymorphisms.

For the receptor-ligand system studied here, LRP-1 ligands (e.g., apoE, apoJ, α₂M) and RAP are not required to bind Aβ. Soluble LRP-1 mutant may bind free Aβ in solution, or with either mutant LRP-1 or Aβ initially attached to a solid phase. After binding between mutant LRP-1 and Aβ, either or both may then be immobilized on a substrate (e.g., cell, tissue, or artificial solid substrate) at any time before, during, or after binding. The mutant LRP-1/Aβ complex may be isolated or detected. Candidate compounds to treat Alzheimer disease may interact with at least one gene, transcript, or protein which is a component of the receptor-ligand system to increase receptor activity (i.e., vascular clearance of Aβ), and be screened for their ability to provide therapy or prophylaxis. These products may be used in assays (e.g., diagnostic methods to detect Aβ using mt-LRP-1) or for treatment; conveniently they are packaged in an assay kit or pharmaceutical form (e.g., single or multiple dose package).

Binding of a soluble LRP-1 mutant directly to Aβ may take place in solution or on a substrate. The assay format may or may not require separation of bound Aβ from unbound Aβ (i.e., heterogeneous or homogeneous formats). Detectable signals may be direct or indirect, attached to any part of a bound complex, measured competitively, amplified, or any combination thereof. A blocking or washing step may be interposed to improve sensitivity and/or specificity. Attachment of the soluble LRP-1 mutant to a substrate before, after, or during binding results in capture of previously unattached receptor. See U.S. Pat. Nos. 5,143,854 and 5,412,087. Abundance may be measured at the level of protein and/or transcripts of a component of the receptor-ligand system.

A soluble LRP-1 mutant may also be attached to a substrate. The substrate may be solid or porous and it may be formed as a sheet, bead, or fiber. The substrate may be made of cotton, silk, or wool; cellulose, nitrocellulose, nylon, or positively-charged nylon; natural rubber, butyl rubber, silicone rubber, or styrenebutadiene rubber; agarose or polyacrylamide; silicon or silicone; crystalline, amorphous, or impure silica (e.g., quartz) or silicate (e.g., glass); polyacrylonitrile, polycarbonate, polyethylene, polymethyl methacrylate, polymethylpentene, polypropylene, polystyrene, polysulfone, polytetrafluoroethylene, polyvinylidenefluoride, polyvinyl acetate, polyvinyl chloride, or polyvinyl pyrrolidone; or combinations thereof. Optically-transparent materials are preferred so that binding can be monitored and signal transmitted by light. Such reagents would allow capture of Aβ in solution by specific interaction between the cognate molecules and then could immobilize Aβ on the substrate.

A soluble LRP-1 mutant may be attached to a substrate through a reactive group as, for example, a carboxy, amino, or hydroxy radical; attachment may also be performed by contact printing, spotting with a pin, pipetting with a pen, or spraying with a nozzle directly onto a substrate. Alternatively, the soluble LRP-1 mutant may be reversibly attached to the substrate by inter-action of a specific binding pair (e.g., antibody-digoxygenin/hapten/peptide, biotin-avidin/streptavidin, glutathione S transferase-glutathione, maltose binding protein-maltose, polyhistidine-nickel, protein A or G/immunoglobulin); crosslinking may be used if irreversible attachment is desired.

Attaching a reporter, which is easily assayed, to a soluble LRP-1 mutant may be used for convenient detection. The reporters may be alkaline phosphatase, β-galactosidase (LacZ), chloramphenicol acetyltransferase (CAT), β-glucoronidase (GUS), bacterial/insect/marine invertebrate luciferases (LUC), green and red fluorescent proteins (GFP and RFP, respectively), horseradish peroxidase (HRP), β-lactamase, and derivatives thereof (e.g., blue EBFP, cyan ECFP, yellow-green EYFP, destabilized GFP variants, stabilized GFP variants, or fusion variants sold as LIVING COLORS fluorescent proteins by Clontech). Reporters would use cognate substrates that are preferably assayed by a chromogen, fluorescent, or luminescent signal. Alternatively, the soluble LRP-1 mutant may be tagged with a heterologous epitope (e.g., FLAG, MYC, SV40 T antigen, glutathione transferase, hexahistidine, maltose binding protein) for which cognate antibodies or affinity resins are available.

A soluble LRP-1 mutant may be joined to one member of the specific binding pair by genetically ligating appropriate coding regions in an expression vector or, alternatively, by direct chemical linkage to a reactive moiety on the binding member by chemical cross-linking. They may be used as an affinity reagent to identify, to isolate, and to detect interactions that involve specific binding with Aβ. This can produce a complex in solution or immobilized to a support.

A mutant LRP-1 may be used as a medicament, diagnostic agent, or used to formulate therapeutic or diagnostic compositions with one or more of the utilities disclosed herein. They may be administered in vitro to a body fluid or tissue in culture, in vivo to a subject's body, or ex vivo to cells outside of the subject that may later be returned to the body of the same subject or another. Fluids and tissues may be further processed after a specimen is taken from the subject's body and before laboratory assay. For example, cells may be diaggregated or lysed, or provided as solid tissue. The specimen may be stored in dry or frozen form prior to assay.

Compounds or derivatives thereof may be used to produce a medicament or other pharmaceutical compositions. Use of compositions which further comprise a pharmaceutically acceptable carrier and compositions which further comprise components useful for delivering the composition to a subject are known in the art. Addition of such carriers and other components to the composition of the invention is well within the level of skill in this art.

The concentration of free Aβ may be decreased by binding to a soluble LRP-1 mutant or removing Aβ bound to a soluble LRP-1 mutant through the body's circulation (e.g., reticuloendothelial system) or by machine (e.g., affinity chromatography, electrophoresis, filtration, precipitation). The efficacy of treatment may be assessed by removal of Aβ from a subject's body or reducing deposition of amyloid in the subject's body. This may be accomplished in a human patient or an animal model where the amount and/or the location of may be detected with a soluble LRP-1 mutant. It should be noted that the modes of treatment described herein differ significantly from the mechanism described in U.S. Pat. No. 6,156,311 that identifies a role for low-density lipoprotein receptor related protein in endocytosis and degradation of amyloid precursor protein (APP).

A label or other detectable moiety may be attached to a soluble LRP-1 mutant or contrast agents may be included for structural imaging: e.g., X-ray computerized tomography (CT), magnetic resonance imaging (MRI), or other optical detection techniques. Functional imaging such as Single Photon Emission Computed Tomography (SPECT) may also be used. A soluble LRP-1 mutant may be labeled (e.g., gadolinium) for MRI evaluation of amyloid load in the brain or vasculature. A soluble LRP-1 mutant may be labeled (e.g., ⁷⁶Br, ¹²³I) for SPECT evaluation of amyloid load in the brain with a blood-brain barrier (BBB) permeabilizing agent, or for evaluating cerebral amyloid angiopathy with or with the BBB permeabilizing agent.

Reagents may also be provided in a kit for use in performing methods such as, for example: diagnosis, identification of those at risk for disease or already affected, or determination of the stage of disease or its progression. In addition, the reagents may be used in methods related to the treatment of disease such as the following: evaluation whether or not it is desirable to intervene in the disease's natural history, alteration of the course of disease, early intervention to halt or slow progression, promotion of recovery or maintenance of function, provision of targets for beneficial therapy or prophylaxis, comparison of candidate drugs or medical regimens, or determination of the effectiveness of a drug or medical regimen. Instructions for performing these methods, reference values and positive/negative controls, and relational databases containing patient information (e.g., genotype, medical history, disease symptoms, transcription or translation yields from gene expression, physiological or pathological findings) are other products that can be considered aspects of the invention.

The amount and extent of treatment administered to a subject in need of therapy or prophylaxis is effective in treating the affected subject. The invention may be used alone or in combination with other known methods. The subject may be any human or animal. Mammals, especially humans and rodent or primate models of disease, may be treated. Thus, both veterinary and medical methods are possible.

A pharmaceutical or diagnostic composition containing one or more mutant LRP-1 protein(s) or nucleic acid(s) encoding the protein(s) may be administered as a formulation adapted for passage through the blood-brain barrier or direct contact with the endothelium. Alternatively, compositions may be added to the culture medium. In addition to the mutant protein or nucleic acid, such compositions may contain physiologically-acceptable carriers and other ingredients known to facilitate administration and/or enhance uptake (e.g., saline, dimethyl sulfoxide, lipid, polymer, affinity-based cell specific-targeting systems). The composition may be incorporated in a gel, sponge, or other permeable matrix (e.g., formed as pellets or a disk) and placed in proximity to the endothelium for sustained, local release. It may be administered in a single dose or in multiple doses which are administered at different times.

A pharmaceutical or diagnostic composition containing one or more mutant LRP-1 protein(s) or nucleic acid(s) encoding the protein(s) may be administered into the body by any known route. By way of example, the composition may be administered by a mucosal, pulmonary, topical, or other localized or systemic route (e.g., enteral and parenteral). The term “parenteral” includes subcutaneous, intradermal, subdermal, intramuscular, intrathecal, intra-arterial, intravenous, and other injection or infusion techniques, without limitation.

Suitable choices in amounts and timing of doses, formulation, and routes of administration can be made with the goals of achieving a favorable response in the subject with Alzheimer disease or at risk thereof (i.e., efficacy), and avoiding undue toxicity or other harm thereto (i.e., safety). Therefore, “effective” refers to such choices that involve routine manipulation of conditions to achieve a desired effect.

A bolus of one or more mutant LRP-1 administered into the body over a short time once a day is a convenient dosing schedule. Alternatively, the effective daily dose of mutant protein(s) or nucleic acid(s) may be divided into multiple doses for purposes of administration, for example, two to twelve doses per day. The dosage of mutant LRP-1 in a pharmaceutical composition can also be varied so as to achieve a transient or sustained concentration in a subject's body, especially in and around vascular endothelium of the brain, and to result in the desired therapeutic response or protection. But it is also within the skill of the art to start doses at levels lower than required to achieve the desired therapeutic effect and to gradually increase the dosage until the desired effect is achieved. Similarly, dosage levels of mutant LRP-1 in a diagnostic composition may be varied to achieve the desired sensitivity and specificity of detection of Aβ in an subject's body.

The amount of mutant LRP-1 administered is dependent upon factors known to skilled artisans such as its bioactivity and bioavailability (e.g., half-life in the body, stability, metabolism); chemical properties (e.g., molecular weight, hydrophobicity, solubility); route (e.g., parenteral, especially intravenous) and scheduling (e.g., frequency per month or year, length of time between successive doses) of the protein's or nucleic acid's administration; and the like. For systemic administration, passage of mutant LRP-1 through the blood-brain barrier is important. It will also be understood that the specific dose level to be achieved for any particular subject may depend on a variety of factors, including age, gender, health, medical history, weight, combination with one or more other drugs, and severity of disease.

The term “treatment” of Alzheimer disease refers to, inter alia, reducing or alleviating one or more symptoms in a subject, preventing one or more symptoms from worsening or progressing, promoting recovery or improving prognosis, and/or preventing disease in a subject who is free therefrom as well as slowing or reducing progression of existing disease. For a given subject, improvement in a symptom, its worsening, regression, or progression may be determined by objective or subjective measures. Efficacy of treatment may be measured as an improvement in morbidity or mortality (e.g., lengthening of survival curve for a selected population). Prophylactic methods (e.g., preventing or reducing the incidence of relapse) are also considered treatment. Treatment may also involve combination with other existing modes of treatment (e.g., ARICEPT or donepezil, EXELON or rivastigmine, anti-amyloid vaccine, mental exercise or stimulation). Thus, combination treatment with one or more other drugs and one or more other medical procedures may be practiced.

The amount of mutant LRP-1 protein(s) or nucleic acid that is administered to a subject is preferably an amount that does not induce toxic or other deleterious effects which outweigh the advantages which result from its administration. Further objectives are to reduce in number, diminish in severity, and/or otherwise relieve suffering from the symptoms of the disease as compared to recognized standards of care. The invention may also be effective against neurodegenerative disorders in general: for example, dementia, depression, confusion, Creutzfeldt-Jakob disease, Huntington's disease, Parkinson's disease, loss of motor coordination, multiple sclerosis, stroke, and syncope.

Production of mutant LRP-1 protein or nucleic acid will be regulated for good laboratory practices (GLP) and good manufacturing practices (GMP) by appropriate governmental regulatory agencies. This requires accurate and comprehensive recordkeeping, as well as monitoring of QA/QC. Oversight of patient protocols by agencies and institutional panels is also envisioned to ensure that informed consent is obtained; safety, bioactivity, appropriate dosage, and efficacy of products are studied in phases; results are statistically significant; and ethical guidelines are followed. Similar oversight of protocols using animal models, as well as the use of toxic chemicals, and compliance with regulations is required.

For therapeutic uses, an appropriate regulatory agency would specify acceptable levels of purity (e.g., lack of extraneous protein and nucleic acids); sterility (e.g., lack of microbes); lack of host cell contamination (e.g., less than 0.5 Endotoxin Unit per mL); and potency (e.g., efficiency of gene transfer and expression) for biologics. Another objective may be to ensure consistent and reproducible production of mutant LRP-1 protein or nucleic acid, which may improve the potency of the biologic while being compatible with the good manufacturing practices used to ensure a pure, sterile, and pyrogen-free product.

Here, direct or indirect interaction between mutant LRP-1 and Aβ at the blood-brain barrier may critically influence neurotoxic and vasculotropic Aβ accumulations by promoting retention of Aβ species with high β-sheet content and genetic mutations within Aβ while clearing soluble Aβ40. Mutations within Aβ do not significantly affect the affinity of mutant Aβ to bind to mt-LRP-1. In contrast to LRP-1, RAGE mediates continuous influx of circulating Aβ into the brain and is overexpressed in brain vasculature in transgenic APP models and in AD (Deane et al., 2003). There is the possibility that mutant LRP-1 action at the blood-brain barrier or in the vascular system will reduce levels of Aβ in the CNS by acting directly to inhibit RAGE-mediated intake of Aβ or indirectly to bind free Aβ in the periphery, thereby resulting in a lower concentration of Aβ in the brain. Applications include subjects with familial forms of Alzheimer disease (FAD) with cerebral amyloid angiopathy (CAA), such as patients with Dutch or Iowa mutations (FAD/CAA). Because mutant LRP-1 binds to both wild-type and mutant Aβ peptide, the mutant LRP-1 can be used for diagnostic purposes in Alzheimer disease, FAD/CAA, and Down syndrome as imaging agents in the brain to visualize changes associated with vascular pathology.

Since the mutant LRP-1 binds Aβ with greater affinity, they can be used to promote egress of Aβ from brain into blood. The levels of Aβ free and bound to soluble LRP-1 mutant can be used to develop an in vitro binding assay (e.g., double-sandwich ELISA blood test) for Alzheimer disease, FAD/CAA, and Down syndrome. The mechanism of action may be sequestration of circulating wild-type or mutant Aβ similar to other peripheral Aβ-binding agents such as anti-Aβ antibody, gelsolin, GM1, and sRAGE. mt-LRP-1 may be used an artificial “sink” that sequesters Aβ in the systemic circulation and prevents Aβ transport across the blood-brain barrier into the brain. Use of one or more mutant LRP-1 protein(s) or nucleic acid(s) provides the advantages that (1) they bind Aβ with greater affinity compared to their wild-type homologs, gelsolin, GM1, sLRP-1 comprising wild-type cluster II and/or cluster IV, or sRAGE and (2) they should be well-tolerated by a subject being treated and thereby avoid an immune or neuroinflammatory response in the brain and cerebral blood vessels because of their smaller size compared to the soluble LRP-1 comprising clusters II and IV.

These properties of mutant LRP-1 can also be used to lower the level of Aβ in the brain of transgenic FAD/CAA mice, other animal models of Alzheimer disease, or Alzheimer disease and FAD/CAA patients by acting as a peripheral sink agent. For this purpose, one or more mutant LRP-1 can be used alone, or together with neuroprotective agents (e.g., activated protein C as described in Guo et al., 2004) or other therapies to lower circulating Aβ in a subject: immunization or vaccination against Aβ; administration of gangliosides, gelsolin, or sRAGE; inhibiting beta/gamma secretase-mediated processing of amyloid precursor protein; osmotic opening of the blood-brain barrier (Neuwelt et al., 1985); normalization of cerebrospinal fluid production (Silverberg et al., 2003); or combinations thereof.

The following examples are merely illustrative of the invention, and are not intended to restrict or otherwise limit its practice.

### EXAMPLES

We screened thirteen mutant LRP-1 and compared them to a soluble derivative of LRP-1 comprising the ligand-binding domain of cluster IV (LRPIV). Seven comprise different fragments of LRPIV without any mutation compared to the native amino acid sequence. Six had point mutations in LRPIV: D3354G, D3394G, D3556G, D3595G, D3633G, and D3674G in SEQ ID NO:1. D3674G in SEQ ID NO:1 corresponds to D343G in cluster IV (SEQ ID NO:3). MT007-LRPIV is the lead compound for the examples below.

LRPIV contains 11 complement related motifs (CR21-CR31), nine of which are calcium-binding fragments, the putative determinants of specific and direct AR binding. Using surface plasmon resonance analysis, CR24-CR28 was shown to be the most effective calcium-binding fragments of LRPIV (Meijer et al., 2007). Three triple-repeats CR24-CR26, CR25-CR27, and CR26-CR28 interact strongly with RAP. CR24-CR26 had the highest binding affinity for activated α2-macroglobulin (α2M*) and factor VIII light chain (FVIII LC), while CR26-CR28 was the best region for factor IXa (FIXa) binding (Meijer et al., 2007). CR23 and CR31 do not appear to contribute to specific and direct Aβ binding. We used CR24-CR28 to produce soluble calcium-binding derivatives of LRPIV and screen for high-affinity Aβ binding. While at least three calcium-binding fragments are required to bind RAP, α2M*, FVIII LC, and FIXa the minimum number of repeats that is required for Aβ binding is unclear. Therefore, LRPIV fragments containing four calcium-binding fragments (CR24-CR27 and CR25-CR28), three calcium-binding fragments (CR25-CR27), two calcium-binding fragments (CR25-CR26 and CR26-CR27) and one calcium-binding fragment (CR25 and CRR26) were produced.

LRPIV comprising all the main ligand binding domains was purified using GST-RAP affinity chromatography. N-terminal amino acid sequence of the purifled LRPIV revealed the presence of three extraneous glycine-alanine-argnine (GAR) amino acids at the N-terminus of the amino acid sequence of LRPIV (GAR-LRPIV). The tPA signal peptide contains a furin cleavage site: _ _ _ _. ⁷RFRRGAR⁻¹ where the endoprotease cuts at RFRR↓GAR, which results in the three extraneous amino acids at the N-terminus of the mutant LRP-1. Exoprotease did not remove the extraneous amino acids. GAR-LRPIV was screened for selective Aβ binding.

Synthesis of cDNA and Cloning. First strand cDNA was synthesized from human spleen total RNA (Clontech) using SuperScript II RT (Invitrogen). Primers were designed based on the human LRP1 sequence (NM_{—}002332). LRPIV domain was amplified from cDNA by polymerase chain reaction (PCR) using Pfx-DNA polymerase (Invitrogen) and their respective primer sets, and cloned into pcDNA3.3 TOPO vector. Using this construct, eleven-repeats of LRPIV (CR21-CR31), two four-repeats (CR24-CR27 and CR25-CR28), one three-repeats (CR25-CR27), two two-repeats (CR25-CR26, CR26-CR27) and two single-repeat derivatives were amplified using PCR and cloned in mammalian expression vector, pSecTag2 B (Invitrogen) in between HindIII and BamHI restriction sites to express soluble protein. pSecTag2 B vector has IgK leader peptide on N-terminal and Myc-tag and His₆-tag on the C-terminus. Full-length secreted LRPIV without any tag (wt-LRPIV), was amplified using 129 bp of forward primer (which has Kozak sequence, start codon, tPA signal peptide sequence and LRPIV sequence) and reverse primer with HindIII restriction site and cloned into pcDNA3.3 TOPO vector. Mutant LRPIV variants were made at six calcium binding sites using Quickchange Lightning Site Directed Mutagenesis kit (Stratagene). WT-LRPIV was used as a template along with their respective primer sets. Insert of the mutated plasmids were sequence verified, restriction digested with SacI and HindIII and cloned into SacI-HindIII digested wt-LRPIV plasmid.

Protein Expression. Suspension Chinese hamster ovary (CHO) cells were grown in CDOpti CHO media supplemented with 1 mM CaCl₂, 2 mM Glutamax at 37° C. on a shaker. CHO cells were stably transfected with each construct using FreeStyle MAX reagent (Invitrogen). Five days after transfection, cells were transferred into media containing antibiotics, 700 μg/mL geneticin for pcDNA 3.3 TOPO or 200 pg/mL hygromycin for pSecTag2. After 12-15 days about 5000 antibiotic resistant cells were plated on 100 mm×10 mm petri plate containing Clone Matrix (Genetix) mixture (40% Clone Matrix, 50% 2× CDOpti CHO, and antibiotics). After about 3 weeks, 50-60 single clones were picked, and transferred into CDOpti CHO media in 48 well plates. After three days, media were tested for expressed LRPIV by Western blot analysis using LRPIV antibody. Selected clones were transferred subsequently into 12 well and 6 well plates. A single selected clone was transferred into flask and grown in suspension culture. LRPIV expression was done in Fernbach flask. Culture was started with 1×10⁶/mL cell density in CDOpti CHO containing 2 mM glutamax, 1 mM CaCl₂, and 10% CHO CD Efficient Feed A (Invitrogen). Each day, cells were counted using hemocytometer (Hausser Scientific Partnership, Horsham, Pa.) and glucose level was measured using GlucCell™ test strip (CESCO Bioengineering Co., Taichung, Taiwan). When the glucose level fell below 2 g/L in the conditioned medium, cells were supplemented with 10% Feed A containing 2 mM glutamax and 1 mM CaCl₂. Usually the feeding was needed after 4 days of culture. Protein was expressed for 10 days, media was harvested by centrifugation and the supernatant was filtered through 0.2 μm filter. Secretion of CR25-CR26, CR26-CR27, CR26, CR27, and mutant variants D3351G, D3592G, D3630G was very low. Therefore, these variants were eliminated from the screening.

Different fragments of LRPIV containing His₆-tag were purified in batch using Ni-NTA agarose (Qiagen). Conditioned media was mixed with 10% glycerol, 150 mM NaCl, 10 mM imidazol and washed Ni-NTA resin, left rocking at room temperature for 30 min and washed with wash buffer (10% glycerol, 300 mM NaCl, 10 mM imidazol and 50 mM NaH₂PO₄, pH 8). Bound protein was eluted with 250 mM imidazol in 50 mM phosphate buffer, pH 8. Eluted protein was passed through 50 KDa cutoff filter (Millipore, Billerica, Mass.). The wt-LRPIV was purified by a single affinity purification step, using GST-RAP affinity column. GST-RAP was expressed, affinity purified using B-PER GST fusion protein purification kit (Pierce) and immobilized on agarose beads using AminoLink Plus coupling Kit (Pierce). Mutant LRPIV variants were purified in one step using anti-LRPIV-antibody affinity column. Anti-LRPIV-antibody column was prepared by immobilizing pure anti-LRPIV antibody to agarose beads using AminoLink Plus coupling kit. About 100 ml of conditioned media was diluted 3× with wash buffer (20 mM Tris, 150 mM NaCl), loaded on anti- LRPIV antibody affinity column, washed with 900 mL of wash buffer, eluted with 0.1M glycine buffer (pH 2.5), neutralized with 2M Tris buffer (pH 9.5) and concentrated using 10 KDa cutoff filter (Millipore). Each purified LRPIV variant was dialyzed against 50 mM carbonate-bicarbonate buffer (pH 9). Their purity was confirmed by silver staining and identify by Western blot analysis.

The single repeats (CR25 and CR26) and double repeats (CR25-26) and CR26-27) were eliminated from the screening due to low expression levels of the proteins and very low binding to Aβ40.

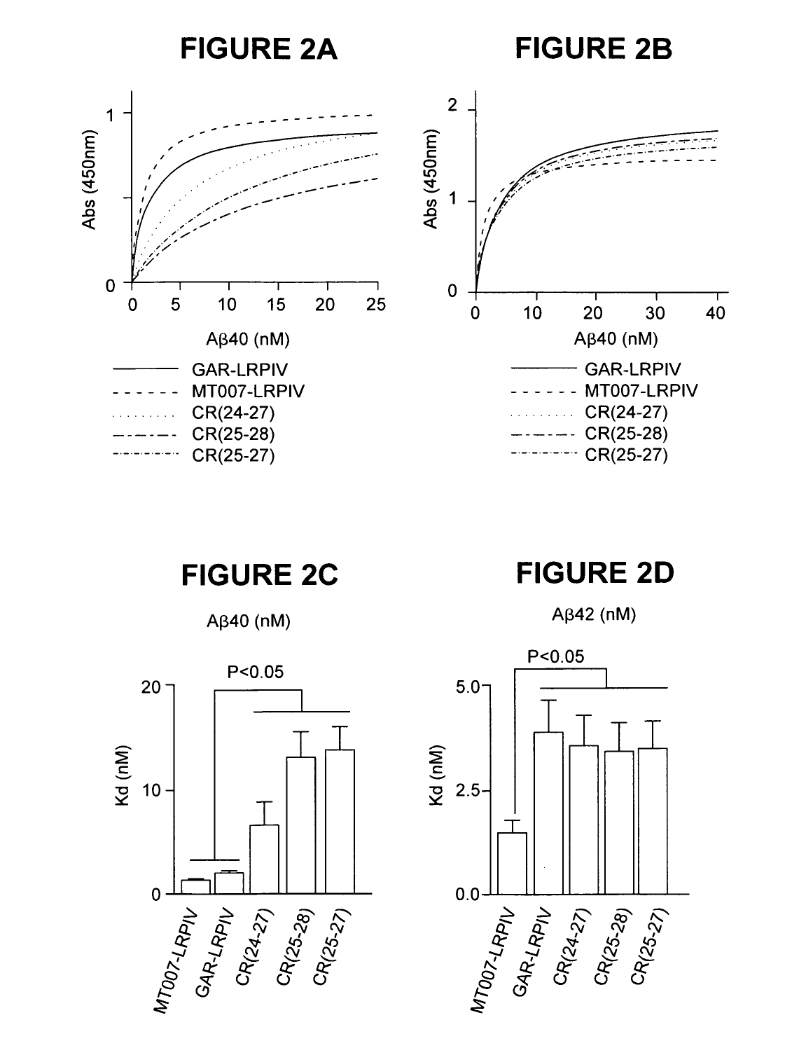

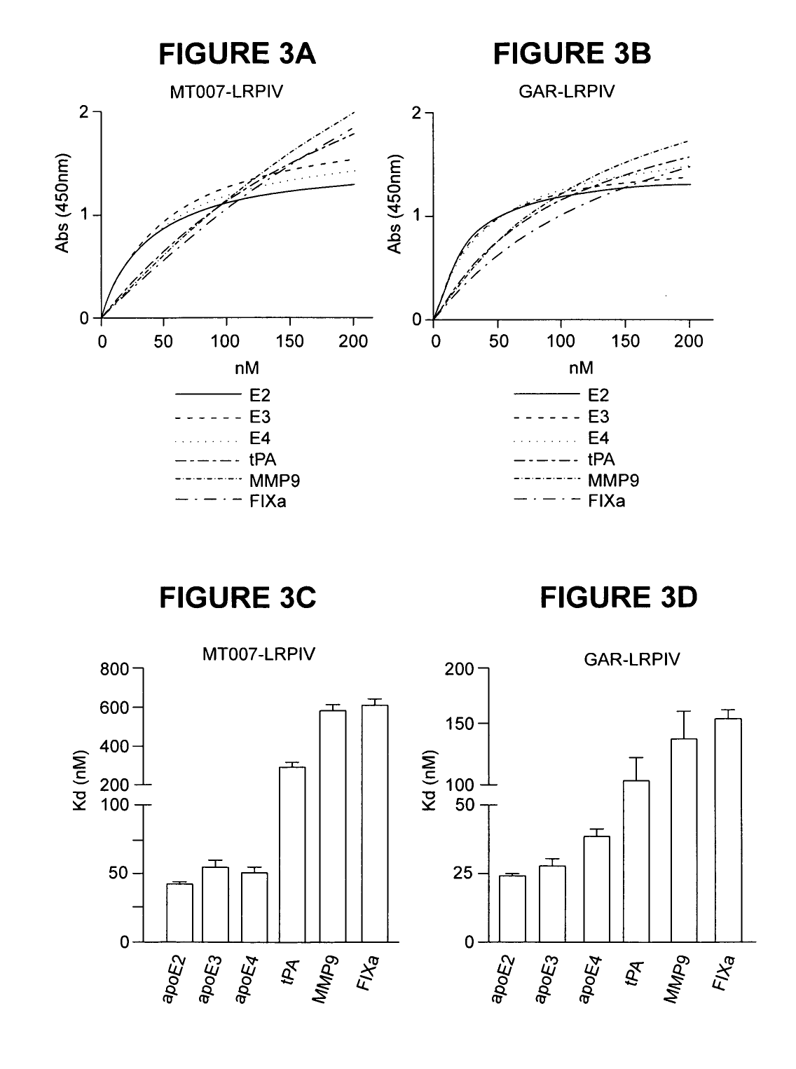

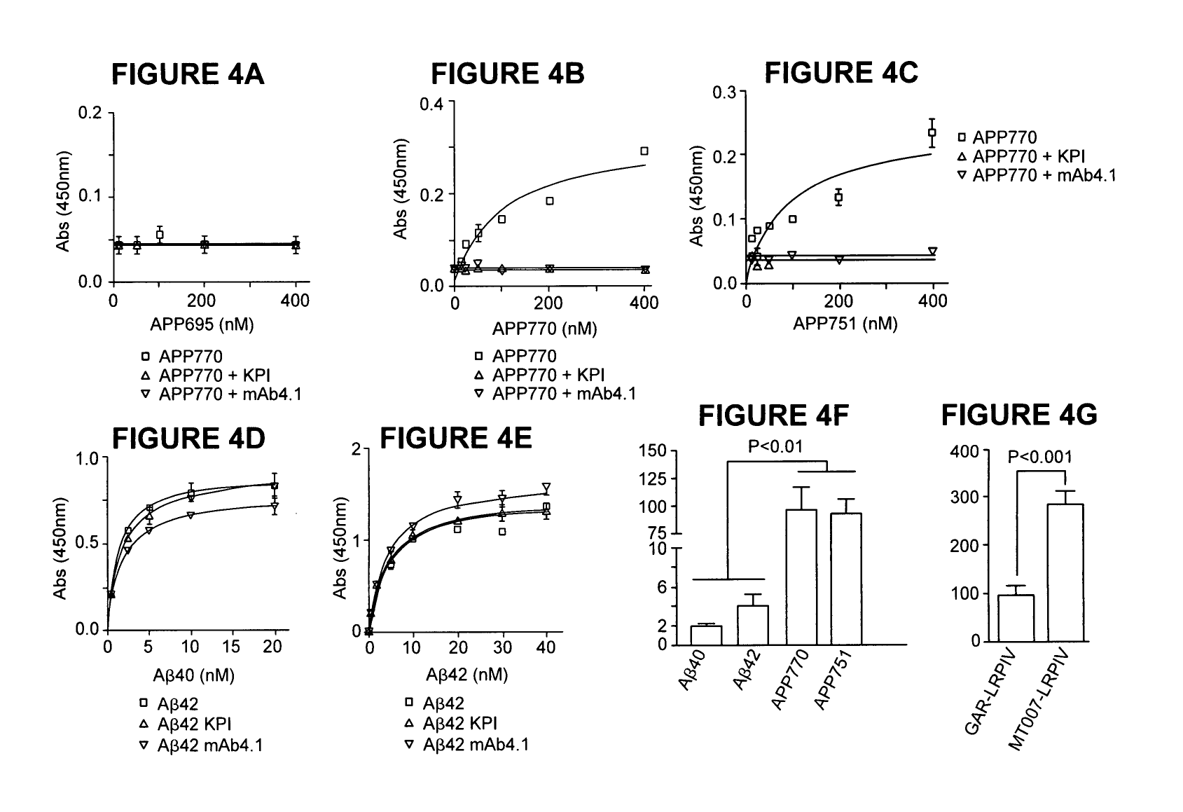

Compared to GAR-LRPIV, the four repeats (CR24-27 and CR25-28) and three repeats (CR25-27) bind Aβ40 with 4- to 8-fold lower affinity (FIGS. 2A-2B). In contrast, compared to GAR-LRPIV, the four and three CRs bind Aβ42 with similar affinity (FIGS. 2C-2D). Compared to GAR-LRPIV, MT007-LRPIV having a mutation in a calcium binding site (D343G), which is outside the region binding RAP, showed selective high affinity binding to Aβ42 and Aβ40 by 2.5- and 1.5-fold, respectively (FIGS. 2A-2D). It is possible that the D343G mutation caused a conformational change in LRPIV that enhanced selective binding of Aβ. Compared to the tagged-LRPIV used by Sagare et al. (2007), MT007-LRPIV had 2.6- and 1.4-fold greater affinity for Aβ42 and Aβ40, respectively. Since LRP-1 interacts with other ligands, we also compared the binding affinities of LRPIV for them. While compared to GAR-LRPIV, MT007-LRPIV binds Aβ42 and Aβ40 with 2.5- and 1.5-fold greater affinity, respectively; it weakly binds the apoEs, with 2-fold lower affinity and tPA, MMP9, and FIXa with 2-, 3- and 4-fold lower affinity, respectively (FIGS. 3A-3D). There was little interaction between α2M* and GAR-LRPIV or MT007-LRPIV. Since LRP-1 interacts with APP via the KPI (Kunitz protease inhibitor) domain (Kounnas et al., 1995), we determined the interaction between GAR-LRPIV and APP isoforms with the KPI domain (APP770, APP751) or without the KPI domain (APP695). APP695 is the major APP isoform in brain. While GAR-LRPIV did not bind to APP695 (FIG. 4A), there was weak binding to APP770 (FIG. 4B) and APP751 (FIG. 4C) that was displaced with soluble KPI domain or with anti-KPI antibody (mAb4.1). But GAR-LRPIV binding to Aβ40 and Aβ42 was not affected by soluble KPI or mAb4.1 (FIGS. 4D-4E). Binding affinities between GAR-LRPIV and APP770 or APP751 were 50- and 25-fold lower than that of Aβ40 and Aβ42, respectively (FIG. 4F). The affinity of binding between APP770 and GAR-LRPIV was 3-fold greater than that between APP770 and MT007-LRPIV (FIG. 4G). Because of the selective and high affinity binding of Aβ40 and Aβ42, MT007-LRPIV was chosen as a lead compound for treating Alzheimer disease by acting as a peripheral sink for Aβ peptides in the brain.

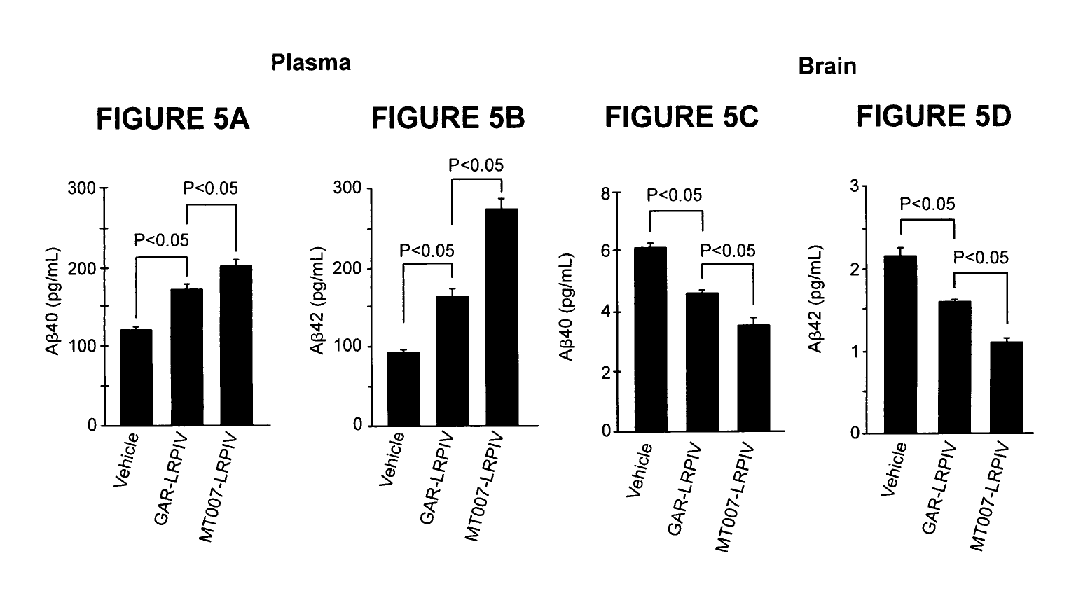

LRPIV in vivo efficacy for lowering the level of brain Aβ. Wild-type mice (2-3 month old C57BL6) were treated with carrier only (vehicle), GAR-LRPIV, or MT007-LRPIV daily (intravenously, 20 μg) for five days. See the similar protocol described in Sagare et al. (2007). At the end of the dosing period, brain tissue and plasma were collected and Aβ levels determined by ELISA. While, compared to vehicle, both LRPIV analogs increased plasma levels of Aβ40 and Aβ42 and decreased their counterparts in brain, the response was significantly greater for MT007-LRPIV (FIGS. 5A-5D). MT007-LRPIV bound significantly more Aβ40 and Aβ42 in plasma (FIGS. 5A and 5B), and was more efficacious in lowering brain Aβ levels than GAR-LRPIV (FIGS. 5C and 5D). To determine how rapid MT007-LRPIV can reduce brain Aβ levels, C57BL6 mice of the same age were treated with a single intravenous bolus of MT007-LRPIV (10 μg) or vehicle. After 12 hrs, Aβ levels in brain and plasma were determined by ELISA. Compared to vehicle, MT007-LRPIV increased plasma Aβ40 and Aβ42 by 1.33- and 2.85-fold, and decreased brain Aβ40 and Aβ42 by 1.68- and 1.45-fold, respectively. Thus, MT007-LRPIV is an effective peripheral sink for Aβ, even for Aβ in the central nervous system.

Immunogenicity of mutant LRP-1. For immunogenicity testing, milligram amounts of mutant LRP-1 protein are needed. Affinity chromatography using a column containing receptor-associated protein (RAP) cannot be used for the isolation of mutant LRPIV proteins that only weakly bind RAP. Since MT007-LRPIV does not bind the GST-RAP column, it was isolated by affinity chromatography using an anti-LRPIV antibody column, which resulted in poor recovery. Therefore, we developed another isolation process using an ion-exchange column that resulted in better purification and yields (see below). Purified protein (2.2 mg) was dialyzed against phosphate buffered saline (PBS) and sent to an outside laboratory for immunogenicity testing. Approximately 8-10 week old BALB/c female mice will be used. Three doses (20, 40, and 80 μg/kg) of MT007-LRPIV were tested. Mice were injected four times bi-weekly. One week after each dose, blood was collected, processed, and tested using antibodies against MT007-LRPIV by ELISA by QED (Bioscience Inc., San Diego, Calif.). There was no immunological response.

MT007-LRPIV protein was expressed in CHO cells. An isolation process may leave the potential for contamination by host cell proteins (HCP) from CHO cells. Therefore, we followed HCP contamination in the final purified protein preparation by two independent methods. (A) Western blot analysis: Samples were separated on SDS-PAGE under reducing conditions, and then transferred to a nitrocellulose membrane. After blocking nonspecific sites, the membrane was exposed to a solution containing goat antibodies raised to CHO protein-free medium. The antibodies were labeled with horseradish peroxidase (HRP). After washing, the protein was detected using an ECL method. The antibodies (Cygnus Technologies) are polyclonal and were generated with broad reactivity to a large number of potential contaminants: i.e., more than 40 different CHO HCP bands from SDS/DTT solubilized CHO cells and from HCP found in conditioned CHO protein free culture media. (B) ELISA: A commercially available kit from Cygnus Technologies was used. It is more sensitive than Western blotting. The kit reacts essentially with all HCP that could contaminate the product independent of purification. The antibodies were generated against affinity purified CHO HCP found in free conditioned medium. No detectable signal was observed by Western blotting. ELISA showed the HCP contamination was less than 100 ppm, which is generally considered acceptable (Cygnus Technologies).

Potential side effects. Studies showed that mice treated with tagged-LRPIV (1 μg/day and 40 μg/kg, intraperitoneally) for three months had no potential side effects (Sagare et al., 2007). Tissue samples and plasma of mice (C57BL6) treated with GAR-LRPIV or MT007-LRPIV (20 pg intravenously, daily for 5 days) were analyzed for potential side effects, but none were observed. There were no significant changes in plasma levels of cholesterol, apoE, tPA, pro-MMP9, and glucose. In a separate group of mice (C57BL6, 2-3 months old) that were dosed at 40 μg/kg, with a single bolus intravenously, blood samples were removed after 2 hr and plasma clotting time was deter-mined as activated partial thromboplastin time (aPTT). This was unchanged. In liver and brain, there were no detectable changes in the expression levels of LDLR or LRP-1. In addition, there were no detectable changes in brain of the level of phosphorrylated LRP-1. Furthermore, APP levels in brain were unchanged by the LRPIV treatment. GAR-LRPIV or MT007-LRPIV did not enter CSF.

### REFERENCES

Dahlgren et al. (2002) Oligomeric and fibrillar species of amyloid-β peptides differentially affect neuronal viability. J. Biol. Chem. 277: 32046-32053.
Deane et al. (2003) RAGE mediates amyloid-β transport across the blood-brain barrier and accumulation in brain. Nature Med. 9: 907-913.
Deane et al. (2004) LRP/Amyloid β-peptide interaction mediates differential brain efflux of AR isoform. Neuron 43: 333-344.
Golabek et al. (1996) The interaction between apolipoprotein E and Alzheimer amyloid beta-peptide is dependent on beta-peptide conformation. J. Biol. Chem. 271: 10602-10606.
Gong et al. (2003) Alzheimer disease-affected brain: Presence of oligomeric Aβ ligands (ADDLs) suggests a molecular basis for reversible memory loss. Proc. Natl. Acad. Sci. USA 100: 10417-10422.
Guo et al. (2004) Activated protein C prevents neuronal apoptosis via protease activated receptors 1 and 3. Neuron 41: 563-572.
Hardy & Selkoe (2002) The amyloid hypothesis of Alzheimer's disease: Progress and problems on the road to therapeutics. Science 297: 353-356.
Herz et al. (1988) Surface location and high affinity for calcium of a 500-kd liver membrane protein closely related to the LDL-receptor suggest a physiological role as lipoprotein receptor. EMBO J. 7: 4119-4127
Kayed et al. (2003) Common structure of soluble amyloid oligomers implies common mechanism of pathogenesis. Science 300: 486-489.
Kounnas et al. (1995) The cellular internalization and degradation of hepatic lipase is mediated by low density lipoprotein receptor-related protein and requires cell surface proteoglycans. J. Biol. Chem. 270: 9307-9312.
Meijer et al. (2007) Functional duplication of ligand-binding domains within low-density lipoprotein receptor-related protein for interaction with receptor associated protein, α2-macroglobulin, factor IXa and factor VIII. Biochim. Biophys. Acta 1774: 714-722.
Neuwelt et al. (1985) Osmotic blood-brain barrier modification: Monoclonal antibody, albumin, and methotrexate delivery to cerebrospinal fluid and brain. Neurosurgery 17: 419-423.
Sagare et al. (2007) Clearance of amyloid-beta by circulating lipoprotein receptors. Nature Med. 13: 1029-1031.
Selkoe (2001) Clearing the brain's amyloid cobwebs. Neuron 32: 177-180.
Silverberg et al. (2003) Alzheimer disease, normal-pressure hydrocephalus, and senescent changes in CSF circulatory physiology: A hypothesis. Lancet Neurol. 2: 506-511.
Van Nostrand et al. (2001) Pathogenic effects of D23N Iowa amyloid β-protein. J. Biol. Chem. 276: 32860-32866.
Walsh et al. (2002) Naturally secreted oligomers of amyloid β protein potently inhibit hippocampal long-term potentiation in vivo. Nature 416: 535-539.
Westein et al. (2002) The α-chains of C4b-binding protein mediate complex formation with low density liporpoprotein receptor-related protein. J. Biol. Chem. 277: 2511-2516.
Wu et al. (2003) A simple method for isolation and characterization of mouse brain microvascular endothelial cells. J. Neurosci. Meth. 130: 53-63.
Zlokovic & Frangione (2003) Transport-clearance hypothesis for Alzheimer's disease and potential therapeutic implications. Aβ Metabolism in Alzheimer's Disease. Ed. T. Saido (Landes Bioscience) 114-122.

Patents, patent applications, books, and other publications cited herein are incorporated by reference in their entirety.

All modifications and substitutions that come within the meaning of the claims and the range of their legal equivalents are to be embraced within their scope. Claims using the transition “comprising” allow the inclusion of other elements to be within the scope of the claim; the invention is also described by such claims using the transition “consisting essentially of” (i.e., allowing the inclusion of other elements to be within the scope of the claim if they do not materially affect operation of the invention) and the transition “consisting” (i.e., allowing only the elements listed in the claim other than impurities or inconsequential activities which are ordinarily associated with the invention) instead of the “comprising” term. For example, “consisting essentially of cluster II and/or cluster IV” would allow the inclusion of other functional domains if the latter did not affect binding of Aβ while “consisting of cluster II and/or cluster IV” would prohibit the inclusion of other functional domains. Any of these three transitions can be used to claim the invention.

It should be understood that an element described in this specification should not be construed as a limitation of the claimed invention unless it is explicitly recited in the claims. In particular, a mutant LRP-1 may be conceived from the native amino acid or nucleotide sequence of LRP-1, preferably human, by deletion to isolate a unit of one or more LRP-1 domain(s), insertion to separate units of one or more LRP-1 domain(s) from each other, fusion to join units of one or more LRP-1 domains with or without extraneous amino acids there-between, and substitution of one or more amino acids or nucleotides in the native sequence. Thus, the granted claims are the basis for determining the scope of legal protection instead of a limitation from the specification being read into the claims. In contradistinction, the prior art is explicitly excluded from the invention to the extent of specific embodiments that would anticipate the claimed invention or destroy novelty.

Moreover, no particular relationship between or among limitations of a claim is intended unless such relationship is explicitly recited in the claim (e.g., the arrangement of components in a product claim or order of steps in a method claim is not a limitation of the claim unless explicitly stated to be so). All possible combinations and permutations of individual elements disclosed herein are considered to be aspects of the invention. Similarly, generalizations of the invention's description are considered to be part of the invention.

From the foregoing, it would be apparent to a person of skill in this art that the invention can be embodied in other specific forms without departing from its spirit or essential characteristics. The described embodiments should be considered only as illustrative, not restrictive, because the scope of the legal protection provided for the invention will be indicated by the appended claims rather than by this specification.

1. A mutant low-density lipoprotein receptor related protein-1 (LRP-1) which binds to amyloid-beta (Aβ) peptide and has at least a mutation of aspartic acid in one or more calcium-binding fragments of LRP-1.

2. The LRP-1 mutant of claim 1, wherein the mutated aspartic acid is preceded by a cysteine within the one or more calcium-binding fragments.

3. The LRP-1 mutant of claim 1, wherein the mutation is substitution of aspartic acid to an amino acid selected from the group consisting of alanine, glycine, serine, and threonine; preferably there is a mutation of aspartic acid (D) to glycine (G).

4. The LRP-1 mutant of claim 1, wherein the mutation is selected from the group consisting of D23G, D64G, D184G, and combinations thereof in LRPII; preferably at least the D184G mutation.

5. The LRP-1 mutant of claim 1, wherein the mutation is selected from the group consisting of D23G, D63G, D143G, D184G, D225G, D264G, D302G, D343G, D386G, and combinations thereof in LRPIV; preferably at least the D343G mutation.

6. The LRP-1 mutant of claim 1 comprising a mutated calcium-binding fragment selected from the group consisting of SEQ ID NO: 4, SEQ ID NO: 5, SEQ ID NO: 8, SEQ ID NO: 12, SEQ ID NO: 13, SEQ ID NO: 15, SEQ ID NO: 16, SEQ ID NO: 17, SEQ ID NO: 18, SEQ ID NO: 19, SEQ ID NO: 20, SEQ ID NO: 21, and combinations thereof; preferably at least SEQ ID NO: 8 and/or SEQ ID NO: 20.

7. The LRP-1 mutant of claim 2 comprising at least three, at least four, at least five, at least six, at least seven, at least eight, or at least nine calcium-binding fragments; preferably 12 or fewer calcium-binding fragments.

8. The LRP-1 mutant of claim 1 comprising SEQ ID NO: 2 and/or SEQ ID NO: 3.

9. The LRP-1 mutant of claim 1 consisting essentially of cluster II and having at least a mutation of aspartic acid in one or more calcium-binding fragments selected from the group consisting of CR3, CR4, and CR7; preferably at least CR7.

10. The LRP-1 mutant of claim 1 consisting essentially of cluster IV and having at least a mutation of aspartic acid in one or more calcium-binding fragments selected from the group consisting of CR21, CR22, CR24, CR25, CR26, CR27, CR28, CR29, and CR30; preferably at least CR29.

11. The LRP-1 mutant of claim 2 which is comprised of at least one domain which mediates secretion.

12. The LRP-1 mutant of claim 2 which is soluble.

13. The LRP-1 mutant of claim 12 which is not comprised of a transmembrane domain.

14. The LRP-1 mutant of claim 1 which is derived from human.

15. The LRP-1 mutant of claim 1 which does not elicit an immune response in human.

16. The LRP-1 mutant of claim 1 further comprising at least one heterologous domain.

17. A composition to inactivate Aβ comprised of (i) a mutant LRP-1 as in claim 1 and (ii) at least one pharmaceuticaly-acceptable carrier.

18. A diagnostic composition to detect Aβ comprised of (i) a mutant LRP-1 as in claim 1 and (ii) at least one detectable label.

19. The diagnostic composition of claim 18, wherein said mutant LRP-1 and said at least one detectable label are covalently attached.

20. The diagnostic composition of claim 18, wherein said mutant LRP-1 and said at least one detectable label are not covalently attached.

21. The diagnostic composition of claim 18, wherein said at least one detectable label is covalently attached to a heterologous domain of said mutant LRP-1.

22. (canceled)

23. A method of binding amyloid-beta (Aβ) peptide in a body fluid and/or tissue of a subject, said method comprising:
(a) providing a soluble low-density lipoprotein receptor related protein-1 (LRP-1) mutant and
(b) contacting said soluble LRP-1 mutant with at least said body fluid and/or tissue of said subject such that said Aβ is specifically bound.

24. The method according to claim 23, wherein said soluble LRP-1 mutant binds said Aβ inside said subject's body.

25. The method according to claim 23, wherein said soluble LRP-1 mutant binds said Aβ outside said subject's body.

26. The method according to claim 23, wherein soluble LRP-1 mutant bound to Aβ is removed from said subject's body.

27. The method according to claim 23, wherein soluble LRP-1 mutant bound to Aβ is inactivated such that there is reduced deposition of amyloid in said subject's body.

28. The method according to 23 further comprising detecting soluble LRP-1 mutant bound to Aβ.

29. The method according to claim 23, wherein Aβ is bound in a body fluid selected from the group consisting of blood, plasma, serum, interstitial fluid (ISF), and cerebrospinal fluid (CSF).

30. The method according to claim 23, wherein Aβ is bound in a tissue selected from the group consisting of brain and other central nervous system tissues, endothelial cells, fibroblasts, smooth muscle cells, and combinations thereof; cerebral arteries, leptomeningial vessels, and temporal arteries; preferably vascular endothelium.

31. The method according to claim 23, wherein said soluble LRP-1 mutant binds to Aβ with at least a ten-fold greater affinity than native LRP-1.

32-35. (canceled)
